(19) United States
(12) Patent Application Publication
(10) Pub. No.: US 2008/0031909 A1
(43) Pub. Date: Feb. 7, 2008

(54) Encapsulated Cosmetic Materials

(75) Inventors: Rudolfus A. Van Benthem, Limbricht (NL); Alexander Poschalko, Birsfelden (CH); Juergen H. Vollhardt, Ramlinsburg (CH)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 11/792,376

(22) PCT Filed: Nov. 30, 2005
(86) PCT No.: PCT/EP05/12752

§ 371 Date: Jul. 11, 2007

(30) Foreign Application Priority Data
Dec. 10, 2004 (EP) 04029375.5

(51) Int. Cl. A61K 8/11; A61Q 17/04; B01J 13/06; B01J 13/20; B01J 13/22

(52) U.S. Cl. 424/401; 424/59; 427/2.14

(57) ABSTRACT

Cosmetic materials, e.g., sunscreen agents are encapsulated by means of a compound of the formula (I) where X is O or NR₅; EWG is an electron-withdrawing group; R₁, R₂, R₃, R₅ are equal to an H, alykl, cycloalkyl, aryl, or heterocyclic group; or R₁, R₂ and R₅ or R₁, R₂ and R₃ together form a heterocyclic group; and wherein at least one NH or NH₂ group, attached to an electron-attracting atom or to an atom that is connected to electron-attracting atom or group such as oxygen, nitrogen and sulphur, is present.

The invention relates to capsules comprising a cosmetic core material, and to a process for their preparation. The invention further relates to the use of such capsules in cosmetic formulations, and to cosmetic formulations containing such capsules.

The term “cosmetic formulation” as used herein refers to compositions which are intended for protecting human skin, hair and teeth against environmental impact and aging processes and maintaining and restoring their normal appearance and function by topical application. Examples of cosmetic formulations are lotions, cremes, gels, lip balms, dentifrices and hair care formulations such as shampoos, conditioners and hair tonics. The term “cosmetic core material” as used herein refers to ingredients which by their physiological action contribute to the desired effect of the cosmetic formulation (hereinafter: cosmetically active agents); and to adjuvants or additives conventionally used in cosmetic or dermatological compositions; but excludes pharmaceutical agents such as antibacterials. The purpose of the capsule can either be a permanent protection of the payload with no or negligible release during application on the skin, or it can be designed to release a cosmetic ingredient in gradually manner over time for a desired long term action, or suddenly to maximum extend after application of the skin. Thickness of the wall size, capsule diameter or the use of co-ingredient to extract a capsule during application are suitable manners to shape the release characteristics as desired. Preferred cosmetic core materials for use in the present invention are cosmetically active agents which comprise UV screening agents, skin anti-aging ingredients, particularily for the protection of sensitive ingredients like e.g.: Vitamin A and derivatives, carotenoides, azulenes, unsaturated fatty acids and derivatives, terpenes, plant extracts, enzymes, but also materials which require a gradual release like e.g. hair growth ingredients or retinol.

Examples of UV screening agents are UV B screening agents, i.e. substances having absorption maxima between about 290 and 320 nm, especially
acrylates such as 2-ethylhexyl 2-cyano-3,3-diphenylacrylate (octocrylene, PARSOL® 340), ethyl 2-cyano-3,3-diphenylacrylate and the like;
camphor derivatives such as 4-methyl benzylidene camphor (PARSOL® 5000), 3-benzylidene camphor, camphor benzalkonium methosulfate, polyacrylamidomethyl benzylidene camphor, sulfobenzylidene camphor, sulfomethyl benzylidene camphor, therephthalidene dicamphor sulfonic acid and the like;
cinnamate derivatives such as octyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes;
p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate,
benzophenones such as benzophenone-3, benzophenone-4,2,2′,4,4′-tetrahydroxy-benzophenone, 2,2′-dihydroxy-4,4′-dimethoxybenzophenone and the like;
esters of benzalmalonic acid such as di (2-ethylhexyl) 4-methoxybenzalmalonate;
esters of 2-(4-ethoxy anilinomethylene)propanedioic acid such as 2-(4-ethoxy anilinomethylene)propanedioic acid diethyl ester as described in EP 895,776;
organosiloxane compounds containing benzmalonate groups as described in EP 358,584, EP 538,431 and EP 709,080;
drometrizole trisiloxane (MEXORYL XL);
pigments such as microparticulated TiO₂, and the like, wherein the term “microparticulated” refers to a particle size from about 5 nm to about 200 nm, particularly from about 15 nm to about 100 nm, more particularly 15 to about 30 nm, and which TiO₂ particles may be coated by metal oxides such as e.g. aluminum or zirconium oxides or by organic coatings such as e.g. polyols, methicones, aluminum stearate, alkyl silane; but particular preferred is the incorporation of uncoated Titanium Dioxide.
imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like.
salicylate derivatives such as isopropylbenzyl salicylate, benzyl salicylate, butyl salicylate, octyl salicylate (NEO HELIOPAN OS), isooctyl salicylate or homomethyl salicylate (homosalate, HELIOPAN) and the like;
triazine derivatives such as octyl triazone (UVINUL T-150), dioctyl butamido triazone (UVASORB HEB), bis ethoxyphenol methoxyphenyl triazine (TINOSORB S) and the like.
   UV A screening agents i.e. substances having absorption maxima between about 320 and 400 nm, especially
dibenzoylmethane derivatives such as 4-tert. butyl-4′-methoxydibenzoyl-methane (PARSOL® 1789), dimethoxydibenzoylmethane, isopropyldibenzoylmethane and the like;
benzotriazole derivatives such as 2,2′-methylene-bis-(6-(2H-benzotriazole-2-yl)-4-(1,1,3,3,-tetramethylbutyl)-phenol (TINOSORB M) and the like;
phenylene-1,4-bis-benzimidazolsulfonic acids or salts such as 2,2-(1,4-phenylene)bis-(1H-benzimidazol-4,6-disulfonic acid) (NEOHELIOPAN AP);
amino substituted hydroxybenzophenones as described in EP 1,046,391 such as 2-(4-diethylamino-2-hydroxy-benzoyl)-benzoic acid hexylester (UVINUL A +);
pigments such as microparticulated ZnO and the like, wherein the term “microparticulated” refers to a particle size from about 5 nm to about 200 nm, particularly from about 15 nm to about 100 nm, more preferred 15 to about 50 nm, and which ZnO particles may be coated by metal oxides such as e.g. aluminum or zirconium oxides or by organic coatings such as e.g. polyols, methicones, aluminum stearate, alkyl silane. Particular preferred is the incorporation of uncoated Zinc Oxide. Also preferred is a mixture of Zinc oxide and Titanium Oxide in the same capsule.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g.,
3,3-diphenylacrylate derivatives as described in EP 514,491 and EP 780,119;
benzylidene camphor derivatives as described in U.S. Pat. No. 5,605,680;
organosiloxanes containing benzalmalonate groups as described in EP 358,584, EP 538,431 and EP 709,080, e.g., dimethico diethylbenzalmalonate (PARSOL SLX).

UV screening agents of particular interest for use in the present invention are octyl methoxycinnamate (PARSOL® MCX) or 4-tert. butyl-4′-methoxydibenzoyl-methane (PARSOL®) 1789), MBC (Merck), and mixtures of Titanium Dioxide and Zinc Oxide pigments.

Examples of carotenoids as core materials for use in the present invention are betacarotene and lycopene. Preferred vitamin A derivatives are retinol and esters thereof, such as alkane carboxylic esters, e.g., the palmitate, propionate, alkyl (e.g. methyl or ethyl) carbonates or acetate; and retinoic acid and esters and amides thereof, such as alkyl retinoates, like e.g. ethylretinoate; or retinoyl-monoalkylamides, e.g. retinoylethylamid; or conjugates of retinoic acid with amino acids. Also, the retinoyl monoethanolamide of which the alcohol group of the ethanolamid function may be ethoxylated, is preferred. Examples of terpenes as core materials for use in the present invention are bisabolol and farnesol.

An example of azulenes as core materials for use in the present invention is chamazulen.

Further examples of cosmetic core materials for use in the present invention are biotin, Coenzyme Q10, and resveratrol.

In one aspect, the invention relates to a process for the preparation of capsules comprising a cosmetic core compound. The process according to that aspect of the invention comprises the steps of:

(1) forming a solution of a compound (1) in a solvent;

(2) forming a dispersion of a cosmetic core material in the solution;

(3) depositing the compound (I) as a resin upon the surface of the core material to form capsules; and

(4) optionally hardening and/or recovering the capsules, whereby steps (1) and (2) are executed in either order or simultaneously, and wherein the compound (I) has the following formula 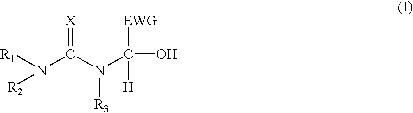
where:
X is O or NR₅;
EWG is an electron-withdrawing group;
R₁, R₂, R₃, R₅ are equal to an H, alkyl, cycloalkyl, aryl, or heterocyclic group; or
R₁, R₂, and R₅ or R₁, R₂, and R₃ together form a heterocyclic group.
   and wherein at least one NH or NH₂ group, attached to an electron-attracting atom or to an atom that is connected to electron-attracting atom or group such as oxygen, nitrogen and sulphur, is present.

Electron-withdrawing groups (EWG) are as such known to the skilled person. Examples of EWG are acid-, ester-, cyano-, di-alkylacetal-, aldehyde-, substituted phenyl-, or trihalomethyl groups. Hydrogen is not an EWG.

Steps (1) and (2) can be carried out in the reversed sequence or in parallel, such that the solution and the dispersion both in the solvent are mixed together. Thus, the description of step (2) as given above should be interpreted to encompass the meaning that a dispersion of the core material is formed in the solvent rather than in the solution, this being the case if step (2) is carried out prior to or simultaneously with step (1).

The first step in the process of the invention is forming a solution of a compound according to formula (I). A compound according to formula (I) is preferably prepared by reacting an amino compound with an aldehyde according to formula (II) or with an aldehyde hydrate according to formula (III) or an alkanol hemiacetal according to formula (IV): 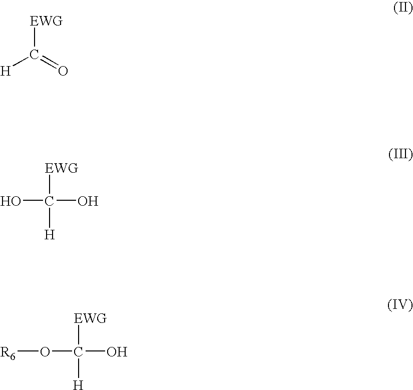
wherein R₆ is C₁-C₁₂alkyl, aryl, aralkyl or cycloalkyl, and EWG is as defined earlier.

Examples of aldehydes according to formula (II) are glyoxilic acid, dimethoxyacetaldehyde, diethoxyacetaldehyde, ethylglyoxylate, butylglyoxylate, and o-phtalaldehyde. Examples of aldehyde hydrates according to formula (III) are glyoxylic acid hydrate, chloral hydrate, and glyoxal hydrate. In formula (IV), R₆ stands for a C₁-C₁₂alkyl group, aryl group, aralkyl group or a cycloalkyl group. Examples of alkanol hemiacetals accoding to formula (IV) are methylglyoxylate methanol hemiacetal and ethylglyoxylate ethanol hemiacetal.

An amino compound is defined herein as a compound having at (east one NH or NH₂ group, attached to an electron-attracting atom or to an atom that is connected to electron-attracting atom or group. The number of amino groups per amino compound generally is at most 3. Examples of electron-attracting atoms are oxygen, nitrogen and sulphur. Suitable amino compounds are for example triazines, guanidine, urea, and mixtures of these compounds. Aminoplasts such as melamine-formaldehyde, urea-formaldehyde and melamine-urea-formaldehyde may also be employed as amino compound. Preferably, urea or triazines such as melamine, melam, melem, ammeline, ammelide and ureidomelamine are used. In particular melamine is used.

The process for the preparation of the compound according to formula (I) will usually occur spontaneously once the amino compound and the compound according to formula (II), (III) or (IV) have been brought into contact with each other. The temperature in the present process can thus vary within wide limits, and preferably lies between 10° C. and 90° C. Most preferably the process is carried out at between 40° C. and 80° C. The process for preparing the amino compound according to formula (I) follows the general rule that it proceeds more quickly if the temperature is raised. An additional control mechanism to influence the reaction rate is the pH, because the addition of either an acid or a base has a catalytic effect. The pH may be adjusted to a value lying preferably between 2 and 10.

Thus, the skilled person can easily—by adjusting temperature and pH—find the circumstances under which a desirable reaction rate is achieved.

The pressure in the present process preferably is between 0.005 MPa and 1.0 MPa, preferably between 0.02 MPa and 0.1 MPa. The process is preferably carried out in a solvent such as for example water or a mixture of water and alkanol. Water is the preferred solvent. Examples of alkanols are methanol, ethanol, propanol, butanol, pentanol.

Starting from the fact that the number of amino groups per amino compound generally is at most 3, the molar ratio between amino group and aldehyde or aldehyde derivative is poreferably between 3 and 1. With more than 3 amino groups per aldehyde or aldehyde derivative, the molecular weight of the resin will be limited, while a ratio below 1 is limiting for crosslinking of the resin and leaves free aldehyde or aldehyde derivative in the solvent.

In a preferred embodiment of the process according to the invention, in step (1) a solution of a compound (V) 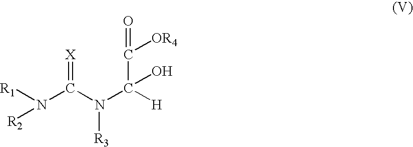
where R₁, R₂, R₃ and X are as defined earlier and R₄ is C₁-C₁₂ alkyl, aryl, aralkyl or cycloalkyl,
and wherein at least one NH or NH₂ group, attached to an electron-attracting atom or to an atom that is connected to electron-attracting atom or group such as oxygen, nitrogen and sulphur in a solvent is formed.

In the compound of formula (V) R₄ is preferably a C₁-C₁₂alkyl group. Examples hereof are methyl, ethyl, propyl, butyl, pentyl, hexyl, heptyl etc. R₄ is in particular a methyl group or an ethyl group. Further preferably, R₃ is hydrogen, and a heterocyclic aminotriazine group is formed by R₁, R₂ and R₅. In a more preferred encapsulated material according to the invention, the aminotriazine ring is derived from melamine.

A compound according to formula (V) is preferably be prepared by reacting an amino compound with an alkanol hemiacetal of formula (IV) above, wherein EWG is —CO—OR₄ where R₄ and R₆ are a C₁-C₁₂ alkyl group, aryl group, aralkyl group or cycloalkyl group, in which process an alkanol is released.

Preferably R₄ and R₆ are C₁-C₁₂ alkyl groups. Examples hereof are methyl, ethyl, propyl, butyl, pentyl, hexyl, heptyl etc. R₄ and R₆ are in particular a methyl group or an ethyl group.

Examples of alkanol hemiacetals of formula (VI) are:

methylglyoxylate methanol hemiacetal (GMHA®, DSM Fine Chemicals, Linz); ethylglyoxylate ethanol hemiacetal (GEHA®, DSM Fine Chemicals, Linz); ethylglyoxylate methanol hemiacetal; butylglyoxylate butanol hemiacetal; butylglyoxylate methanol hemiacetal; butylglyoxylate ethanol hemiacetal; isopropylglyoxylate isopropanol hemiacetal; propylglyoxylate propanol hemiacetal; cyclohexylglyoxylate methanol hemiacetal and 2-ethylhexylglyoxylate methanol hemiacetal. It is also possible to use ethyl or butyl glyoxylate in stead of the hemiacetal.

The second step in the process of the invention is forming a dispersion of a core material in the solution. If the core is a first liquid, the material to be encapsulated can be this first liquid. The core material can also be a solid or a second liquid which is dissolved or dispersed in said first liquid. Said first liquid preferably is a high boiling hydrophobic liquid such as an oil. Suitable oils, are in particular vegetable and animal oils, fatty esters and waxes, partly hydrogenated terphenyls, chlorinated paraffins, alkylated biphenyls, alkyl naphthalenes, diaryl methane derivatives, dibenzyl benzene derivatives, alkanes, cycloalkanes and esters, such as phthalates, adipates, trimellitates and phosphates, and silicone oils.

To stabilize the dispersion a surfactant can be added. Suitable surfactants can be found among ionic and non-ionic surfactants. The surfactant preferably is an anionic or non-ionic surfactant. It is not always necessary to use such a surfactant, since many of the compounds according to formula (VI) spontaneously form small amounts of anionic groups through hydrolysis which can act as a surfactant.

The third step in the process of the invention is depositing the compound as a resin upon the surface of the core material to form capsules. Step (3) generally involves changing the conditions in such a way as to cause phase separation of the wall material from the continuous wall solution phase. Normally, the wall forming material is caused to phase separate from the continuous phase, at least partially as a coherent film around the particles or droplets of the core phase in a process which preferably lasts between several minutes and hours. Phase separation can be introduced by an increase or decrease of the temperature. A decrease of temperature may cause phase separation due to a decreased solubility, while an increase of the temperature may cause the resin to pass over its cloud point.

An alternative way of phase separation is to increase the molecular weight of the resin. This is effected by prolonged polymerization of the compound according to formula (I) or (V) in the solvent. This will decrease the solubility of the resin in the solvent.

A third way to introduce phase separation is to increase or to decrease the concentration of the resin, thus using the fact that resins from compounds according to formula (I) or (V) generally have a range of maximum solubility.

Since it is the purpose of the process according to the invention to form capsules, a high percentage of the core material should be fully encapsulated in the third step; preferably, at least 80 wt. % or 85 wt. % of the core material is fully encapsulated in the third step, more preferable at least 90 wt. %, in particular at least 95 or even 99 wt. %; most preferably, essentially all core material is fully encapsulated in the third step.

The optional forth step in the process of the invention is the hardening and isolation of the capsules. In this case the liquid or gelatinous wall phase is preferably hardened, before isolation of the capsules. Hardening can be done by lowering the temperature below the T_{g} of the resin, or by polymerisation of the resin in order to obtain an elastic non-sticky capsule. In a preferred embodiment of the process according to the invention, hardening is incorporated into the third step. Capsule recovery can be effected by for example filtering or centrifuging, optionally followed by drying or spray drying in case the capsules are to be recovered as a dry powder. In some instances, the dried product is a caked powder and must be reduced to a free flowing powder by a gentle grinding operation, e.g., sieving.

In another aspect, the invention relates to the use of the capsules of the present invention as a component in cosmetic formulations and to cosmetic formulations containing such capsules. Depending on the nature of the core material, it may be desirable or required to release the core material from the capsules. This is typically achieved by mechanical stress when applying the cosmetic formulation on the skin. While the capsules of the pre-sent invention preferably have a size of from about 1 μm to about 200 μm, the size of the capsules is suitably adjusted from about 10 μm to about 30 μm when release of the core material from the capsule by mechanical stress is desired. When release of the core material from the capsule is not necessarily required, e.g., when the core material is a UV screening agent which exerts the desired activity also while encapsulated the capsules may be of smaller size, e.g. of from about 1 μm to about 3 μm. Capsules of larger size, e.g. from about 100 μm to about 200 μm may be prepared to attain a decorative effect to the formulation or to provide an abrasive effect on the skin. The size of the capsules can be adjusted by the appropriate choice of the shearing force in the dispersion step (2) of their preparation. Thus, a capsule size of from about 1 μm to about 3 μm can be achieved by using a high-pressure homogenizer. A capsule size of from about 10 μm to about 30 μm can be obtained by using a high-speed homogenizer, such as an ULTRA-TURRAX homogenizer. Larger capsules, e.g. of a size of from about 100 μm to about 200 μm are suitably obtained by low-shear stirring in the dispersion step (2). Furthermore, the choice of the emulsifier used may exert an impact on the capsule size. It will be appreciated that the parameters required to obtain a desired capsule size are within the skill of the expert.

The thickness of the encapsulating wall can be adapted to the requirements of the specific application, e.g., to control the release, if required, of the core material from the capsules. A simple way to control the wall thickness is by choosing the concentration of the mixture in the solvent taking into account the particle size in relation to the wall thickness. The weight ratio of the mixture and the solvent generally is between 0.06 and 0.8, whereby the precise ranges strongly depend on the solubility of the specific compound according to formula (I) or (V) used. Compounds according to formula (I) or (V) typically have a maximum solubility within the above mentioned range. The precise range for a particular compound can easily be established by a person skilled in the art.

The invention further relates to an encapsulated material comprising a core material and a wall material, wherein the wall material comprises a resin prepared from a compound according to formula (I) of claim 1. In a preferred embodiment, the compound according to formula (I) is an amino compound according to formula (V) wherein a heterocyclic aminotriazine group is formed by R₁, R₂ and R₅, and wherein R₃ is H and R₄ is methyl or ethyl. In a more preferred encapsulated material according to the invention, the aminotriazine ring is derived from melamine.

The capsules of the present invention may be formulated into cosmetic vehicles as such or in combination with the non encapsulated core material for an additional effect. It is also possible to combine the capsules with other cosmetic ingredients like. emollients, emulsifiers, co-emulsifiers, humectants, vitamins, other skin care actives, preservatives, moisturizing factors, etc. The amount of capsules in the final cosmetic composition is adjusted to the amount of core material required to be present in the formulation.

The cosmetic compositions of the invention are useful e.g. as compositions for photoprotecting the human epidermis or hair against the damaging effect of ultraviolet irradiation, as sunscreen compositions and as skin anti-aging compositions. Such compositions can, in particular, be provided in the form of a lotion, a thickened lotion, a gel, a cream, a milk, an ointment, a powder, a spray, a foam or a solid tube stick and can be optionally be packaged as an aerosol and can be provided in the form of a mousse, foam or a spray. When the cosmetic composition according to the invention are provided for protecting the human epidermis against UV radiation or as sunscreen composition, they can be in the form of a suspension or dispersion in solvents or fatty substances, or alternatively in the form of an emulsion or microemulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactants. The manufacture of such cosmetic compositions can be accomplished by technologies which are known per se to one skilled in the art.

The invention is further elucidated by the following non-limiting examples.

### EXAMPLE I

An emulsion of 50 g ethylhexyl 4-methoxy cinnamate in 150 ml water containing 2% Luviskol K90 (BASF) was prepared by homogenization with an ULTRA-TURRAX® mixing device at 24'000 rpm. Separately, a mixture of 11.2 g melamine, 15.8 g glyoxylate (GMHA) and 12 g water were stirred at 80° C. to give a clear resin solution. The UV-filter emulsion was then added to the resin solution with continous stirring, and the temperature maintained at 60° C. for 4 hours. In this fashion, hardening of the capsules was achieved by extended reaction of the wall material. The resulting suspension was cooled to room temperature.

### EXAMPLE II

A solution of 20.0 g retinyl acetate and 2.0 g BHT in 30.0 g Tegosoft® TN was purged with Argon for 15 min. Separately, 150 mL water containing 2% Luviskol K90 (BASF) were purged with Argon for 15 min. The solutions were mixed together and homogenized with an ULTRA-TURRAX® mixing device at 24'000 rpm to give a slightly yellow emulsion. Separately, a mixture of 11.2 g melamine, 15.8 g glyoxylate (GMHA) and 12 g water were stirred at 80° C. to give a clear resin solution. The emulsion was then added to the resin solution with continuous stirring. The temperature was maintained at 60° C. for 4 hours to harden the formed capsules by extended reaction of the wall material. The resulting slightly yellow suspension was cooled to room temperature and stored until further use at 4° C. in the dark.

### EXAMPLE III

### Preparation of an Amphisol K Formulation Containing 5% (w/w) of Encapsulated Chromophor as a o/w Sunscreen

A mixture of 2.0 g Amphisol K, 3.0 g Estol GMM 3650, 1.0 g Cetyl Alcohol, 14.0 g Miglyol 812 N, 0.05 g BHT, and 1.0 g Phenonip was shortly heated to 80° C. in order to melt solid emulsifiers. To the still warm mixture (70-80° C.) a preheated solution (˜80° C.) of 5.0 g glycerin, 0.1 g EDTA BD, and 0.2 g 10% aqueous KOH in 49.85 g water was added slowly under continuous stirring. The resulting emulsion was stirred until a temperature of about 40° C. was reached and homogenized with 24'000 rpm using a ULTRATURRAX® mixing device. 23.8 g suspension of encapsulated Ethylhexyl 4-methoxy cinnamate as obtained in Example I were added under continuous stirring. The resulting sunscreen was finally stirred until room temperature was reached.

### EXAMPLE IV

A hair tonic can be prepared from the constituents indicated below.

| | | |
|---|---|---|
| Water demineralized | | ad.to |
| | | 100.00 |
| Stay-C 50 | (Sodium Ascorbyl Phosphate) | 0.20 |
| Ethanol | | 30.00 |
| 2-Propanol | | 10.00 |
| D-Panthenol 75 L | (Panthenol) | 0.50 |
| Edeta BD | (Disodium EDTA) | 0.20 |
| Dow Corning 193 | (PEG-12 Dimethicone) | 0.20 |
| Surfactant | | |
| Luviskol VA64 Powder | (VP/VA Copolymer) | 3.00 |
| Cremophor RH 410 | (PEG-40 Hydrogenated Castor Oil) | 1.00 |
| Phytantriol | | 0.10 |
| dl-alpha-Tocopheryl | | 0.20 |
| Acetate | | |
| d-Biotin encapsulated | | 0.05-10% |
| in analogy to the | | |
| procedure of Example I | | |

### EXAMPLE V

A Sun Milk can be prepared from the constituents indicated below.

| | | |
|---|---|---|
| Parsol SLX | (Polysilicone-15) | 5.00 |
| Uvinul TiO2 | (Titanium Dioxide) | 2.00 |
| Uvinul A+ encapsulated | (Diethylamino Hydroxybenzoyl | 0.5-20.00 |
| in analogy to the | Hexyl Benzoate) | |
| procedure of Example I | | |
| Cosmacol ESI | (Tridecyl Salicylate) | 8.00 |
| Dow Corning 2503 | (Stearyl Dimethicone) | 2.00 |
| Cosmetic Wax | | |
| Myritol 318 | (Caprylic/Capric Triglyceride) | 6.00 |
| Lorol C 16 | (Cetyl Alcohol) | 1.00 |
| Butylated Hydroxy- | | 0.05 |
| toluene | | |
| Phenonip | (Phenoxyethanol & Methylparaben | 0.80 |
| | & Ethylparaben & Butylparaben & | |
| | Propylparaben & Isobutylparaben) | |
| Estol 3650 | (Glyceryl Myristate) | 4.00 |
| Amphisol K | (Potassium Cetyl Phosphate) | 2.00 |
| Glycerol | | 3.00 |
| Edeta BD | (Disodium EDTA) | 0.10 |
| Carbopol Ultrez 21 | (Acrylates/C10-30 Alkyl Acrylate | 0.30 |
| | Cross-polymer) | |
| Water demineralized | | add to |
| | | 100.00 |
| Water demineralized. | | 20.00 |
| Parsol HS | (Phenylbenzimidazole Sulfonic | 2.00 |
| | Acid) | |
| Potassium Hydroxide | | 0.55 |

### EXAMPLE VI

A Night Cream can be prepared from the constituents indicated below.

| | | |
|---|---|---|
| Cremophor WO-7 | (PEG-7 Hydrogenated Castor Oil) | 6.00 |
| Elfacos ST-9 | (PEG-45/Dodecyl Glycol | 2.00 |
| | Copolymer) | |
| Paracera M | (Microcrystalline Wax) | 2.00 |
| Myritol 318 | (Caprylic/Capric Triglyceride) | 5.00 |
| Jojoba Oil | (Simmondsia Chinensis (Jojoba) | 5.00 |
| | Seed Oil) | |
| Paraffin Oil | | 5.00 |
| Butylated Hydroxy- | | 0.05 |
| toluene | | |
| Phenonip | (Phenoxyethanol & Methylparaben | 0.80 |
| | & Ethylparaben & Butylparaben & | |
| | Propylparaben & Isobutylparaben) | |
| Dow Corning | (Dimethicone) | 0.50 |
| 200/350 cs | | |
| Glycerol | | 3.00 |
| Sodium Chloride | | 0.70 |
| Retinol encapsulated | | 0.1-20.00 |
| in analogy to the | | |
| procedure of Example | | |
| II | | |
| Edeta BD | (Disodium EDTA) | 0.10 |
| Citric Acid, Anhydrous | | 0.25 |
| Water demineralized | | ad. 100.00 |
| Water demineralized | | 15.00 |
| Citric Acid, Anhydrous | | 0.25 |

### EXAMPLE VII

A Lip Balm can be prepared from the constituents indicated below.

| | | |
|---|---|---|
| Parsol SLX | (Polysilicone-15) | 5.00 |
| Parsol 1789 | (Butyl Methoxydibenzoylmethane) | 0.2-20.00 |
| encapsulated in | | |
| analogy to the | | |
| procedure of | | |
| Example I | | |
| Eutanol G | (Octyldodecanol) | 22.40 |
| Castor Oil | | Add to 1000 |
| Myritol 318 | (Caprylic/Capric Triglyceride) | 18.00 |
| Softisan 649 | (Bis-Diglyceryl Polyacyladipate-2) | 7.00 |
| Syncrowax HR-C | (Tribehenin) | 2.00 |
| Carnauba Wax 2442 | (Copernicia Cerifera Wax) | 8.00 |
| Beeswax | | 10.00 |
| dl-alpha-Tocopheryl | | 2.00 |
| Acetate | | |
| Jojoba Oil | (Simmondsia Chinensis (Jojoba) Seed | 1.00 |
| | Oil) | |
| Alpha-Bisabolol | | 0.20 |
| D-Panthenol | | 0.20-10.00 |

### EXAMPLE VIII

A Safe Sun Milk can be prepared from the constituents indicated below.

| | | |
|---|---|---|
| Estol 3650 | (Glyceryl Myristate) | 2.50 |
| Lorol C 16 | (Cetyl Alcohol) | 2.50 |
| Parsol MCX encapsulated | (Ethylhexyl | 0.1-40.00 |
| in analogy to | Methoxycinnamate) | |
| the procedure of Example I | | |
| Parsol 1789 encapsulated | (Butyl | 0.1-30.00 |
| encapsulated | Methoxydibenzoylmethane) | |
| in analogy to | | |
| the procedure of Example I | | |
| Parsol 340 encapsulated | (Octocrylene) | 0.1-30.00 |
| in analogy to | | |
| the procedure of Example I | | |
| Macadamia Seed Oil | | 2.00 |
| Finsolv TN | (C12-15 Alkyl Benzoate) | 4.00 |
| Butylated Hydroxy- | | 0.05 |
| toluene | | |
| Dow Corning | (Dimethicone) | 0.50 |
| 200/350 cs | | |
| dl-alpha-Tocopheryl | | 2.00 |
| Acetate | | |
| Phenonip | (Phenoxyethanol & | 0.80 |
| | Methylparaben & | |
| | Ethylparaben & Butylparaben | |
| | & Propylparaben & | |
| | Isobutylparaben) | |
| Brij 72 | (Steareth-2) | 2.00 |
| Brij 721 | (Steareth-21) | 2.00 |
| Glycerol | | 3.00 |
| Edeta BD | (Disodium EDTA) | 0.10 |
| Keltrol CG T | (Xanthan Gum) | 0.20 |
| Carbopol Ultrez 21 | (Acrylates/C10-30 Alkyl | 0.25 |
| | Acrylate Cross-polymer) | |
| Water demineralized | | add. to |
| | | 100.00 |
| Stay C 50 | (Sodium Ascorbyl Phosphate) | 0.10-25.00 |

### EXAMPLE IX

A Lip Balm can be prepared from the constituents indicated below.

| | | |
|---|---|---|
| Parsol SLX | (Polysilicone-15) | 5.00 |
| Parsol 1789 encapsulated | (Butyl | 0.2-20.00 |
| in analogy to | Methoxydibenzoylmethane) | |
| the procedure of Example I | | |
| Eutanol G | (Octyldodecanol) | 22.40 |
| Castor Oil | | Add to 1000 |
| Myritol 318 | (Caprylic/Capric Triglyceride) | 18.00 |
| Softisan 649 | (Bis-Diglyceryl | 7.00 |
| | Polyacyladipate-2) | |
| Syncrowax HR-C | (Tribehenin) | 2.00 |
| Carnauba Wax 2442 | | 8.00 |
| Beeswax | | 10.00 |
| dl-alpha-Tocopheryl | | 2.00 |
| Acetate | | |
| Jojoba Oil | | 1.00 |
| Alpha-Bisabolol | | 0.20 |
| D-Panthenol | | 0.20-10.00 |

### EXAMPLE X

An Eye Cream can be prepared from the constituents indicated below.

| | | |
|---|---|---|
| Estol 3650 | (Glyceryl Myristate) | 2.50 |
| Lorol C 16 | (Cetyl Alcohol) | 2.50 |
| Coenzyme Q10 encapsulated | | 0.01-10.00 |
| in analogy | | |
| to the procedure | | |
| of Example I | | |
| Parsol 1789 encapsulated | (Butyl | 0.5-20.00 |
| in analogy to | Methoxydibenzoylmethane) | |
| the procedure of Example I | | |
| Resveratrol encapsulated | | 0.01-10.00 |
| in analogy to | | |
| the procedure of Example I | | |
| Retinol encapsulated | | 0.01-10.00 |
| in analogy to the | | |
| procedure of Example | | |
| II | | |
| Jojoba Oil | | 2.00 |
| Myritol 318 | (Caprylic/Capric Triglyceride) | 15.00 |
| Butylated Hydroxy- | | 0.05 |
| toluene | | |
| dl-alpha-Tocopheryl | | 2.00 |
| Acetate | | |
| Phenonip | (Phenoxyethanol & | 0.80 |
| | Methylparaben & | |
| | Ethylparaben & Butylparaben | |
| | & Propylparaben & | |
| | Isobutylparaben) | |
| Dow Corning | (Dimethicone) | 0.50 |
| 200/350 cs | | |
| Brij 72 | (Steareth-2) | 2.00 |
| Brij 721 | (Steareth-21) | 2.00 |
| 1,3-Butylenglykol | | 2.00 |
| Glycerol | | 3.00 |
| Edeta BD | (Disodium EDTA) | 0.10 |
| Keltrol CG T | (Xanthan Gum) | 0.20 |
| Carbopol Ultrez 21 | (Acrylates/C10-30 Alkyl | 0.25 |
| | Acrylate Cross-polymer) | |
| Hyasol-BT | (Sodium Hyaluronate) | 5.00 |
| Water demineralized | | add. to |
| | | 100.00 |

1. Process for forming capsules comprising the steps of:
(1) forming a solution of a compound (I) in a solvent;
(2) forming a dispersion of a cosmetic core material in the solution;
(3) depositing the compound (I) as a resin upon the surface of the core material to form capsules; and
(4) optionally hardening and/or recovering the capsules,
whereby steps (1) and (2) are executed in either order or simultaneously, and wherein the compound (I) has the following formula 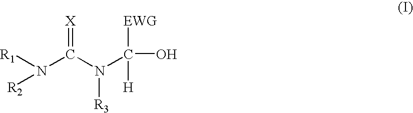
where:
X is O or NR₅;
EWG is an electron-withdrawing group;
R₁, R₂, R₃, R₅ are equal to an H, alkyl, cycloalkyl, aryl, or heterocyclic group; or
R₁, R₂, and R₅ or R₁, R₂, and R₃ together form a heterocyclic group
and wherein at least one NH or NH₂ group, attached to an electron-attracting atom or to an atom that is connected to electron-attracting atom or group such as oxygen, nitrogen and sulphur, is present.

2. Process according to claim 1, wherein in step (1) the compound (I) is formed by reacting an amino compound with an aldehyde of the formula (II) or with an aldehyde hydrate of the formula (III) or an alkanol hemiacetal of the formula (IV): 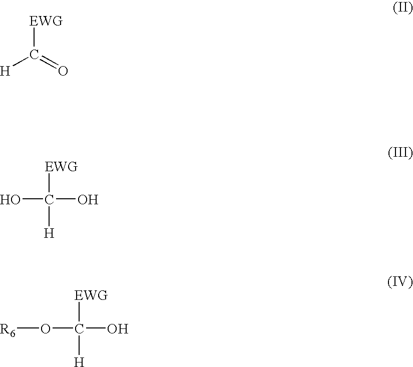
wherein R₆ is C₁C₁₂ alkyl, aryl, aralkyl or cycloalkyl, and EWG is as defined earlier.

3. Process according to claim 2, wherein EWG is an acid-, ester-, cyano-, di-alkylacetal-, aldehyde-, substituted phenyl-, or trihalomethyl group.

4. Process according to claim 3 wherein the compound of formula (I) is formed by reacting an amino compound with a compound of formula (IV) wherein EWG is a group —CO—OR₄, and R₄ is methyl or ethyl.

5. Process according to claim 2 wherein in the amino compound the number of NH or NH₂ groups is 1 to 3.

6. Process according to claim 5 wherein the amino compound is a triazine, guanidine, urea, or mixtures of these compounds, or an aminoplast such as melamine-formaldehyde, urea-formaldehyde and melamine-urea-formaldehyde.

7. Process according to claim 5 wherein the amino compound is urea or a triazine.

8. Process according to claim 5 wherein the amino compound is melamine, melam, melem, ammeline, ammelide or ureidomelamine, preferably melamine.

9. Process according to claim 1, wherein the solvent is water.

10. Process according to claim 9, wherein the molar amino group/hemiacetal ratio is between 3 and 1.

11. Process according to claim 1 wherein the cosmetic core material is at least one of a cosmetically active agent or an adjuvant or additive conventionally used in cosmetic or dermatological compositions.

12. Process according to claim 11 wherein the cosmetic core material is a cosmetically active agent selected from a UV screening agent, a carotenoid, a vitamin, a skin care active, a terpene, or azulene.

13. Process according to claim 11 wherein the cosmetic core material is TiO₂ or ZnO.

14. Process according to claim 11 wherein the cosmetic core material is PARSOL MCX.

15. Process according to claim 11 wherein the cosmetic core material is retinol.

16. Capsules comprising a cosmetic core material and a wall material, characterized in that the wall material comprises a resin prepared from a compound according to formula (I) of claim 1.

17. Capsules according to claim 16, wherein in the compound according to formula (I) EWG is —COOR₄, a heterocyclic aminotriazine group is formed by R₁R₂ and R₅, and wherein R₃ is H and R₄ is methyl or ethyl.

18. Capsules according to claim 17, wherein the aminotriazine ring is derived from melamine.

19. Capsules according to claim 16, wherein the core material is at least one of a cosmetically active agent or conventionally used in cosmetic or dermatological compositions.

20. Capsules according to claim 16, wherein the cosmetic core material is a cosmetically active agent selected from a UV screening agent, a carotenoid, a vitamin, a skin care active, a terpene, or azulene.

21. Capsules according to claim 20 wherein the cosmetic core material is TiO₂ or ZnO.

22. Capsules according to claim 20 wherein the cosmetic core material is PARSOL MCX.

23. Capsules according to claim 20 wherein the cosmetic core material is retinol.

24. The use of capsules according to claim 16 as a component in cosmetic formulations

25. Cosmetic formulations comprising capsules according to claim 16.

26. Cosmetic formulations according to claim 25 which are a lotion, a creme, a gel or a shampoo.
